(19) United States
(12) Patent Application Publication
(10) Pub. No.: US 2025/0115941 A1
(43) Pub. Date: Apr. 10, 2025

(54) Exposure device for exposing microorganisms to gaseous pollutants and detection system thereof

(71) Applicant: FUDAN UNIVERSITY, Shanghai (CN)

(72) Inventors: Dan LI, Shanghai (CN); Shuo YANG, Shanghai (CN); Xiang ZHANG, Shanghai (CN); Jianmin CHEN, Shanghai (CN)

(73) Assignee: FUDAN UNIVERSITY, Shanghai (CN)

(21) Appl. No.: 18/885,575

(22) Filed: Sep. 14, 2024

(30) Foreign Application Priority Data
Sep. 17, 2023 (CN) 202311195397X
Sep. 17, 2023 (CN) 2023111981486
Sep. 17, 2023 (CN) 2023111981626

(51) Int. Cl. C12Q 1/02; C12M 1/00; C12M 1/02; C12M 1/34; C12M 3/06

(52) U.S. Cl. CPC C12Q 1/025; C12M 23/16; C12M 23/40; C12M 29/08; C12M 41/18; C12M 41/46

(57) ABSTRACT

An exposure device for exposing microorganisms to gaseous pollutants and a corresponding in-situ multi-toxicity endpoint detection system are provided. The detection system based on the exposure device disclosed in the present application may perform controlled accumulation of pollutants in air, and implements in-situ, comprehensive, quick, and low-cost assessment of toxicity effects of pollutants in air of a target site. The exposure device and the detection system disclosed in the present application can effectively avoid toxicity distortion caused by complex collection and transfer process, complex chemical reactions in a liquid elution process in conventional toxicity detection methods for gaseous pollutants.

### FIELD OF TECHNOLOGY

The present application belongs to the technical field of environmental protection, and particularly relates to an exposure device for exposing microorganisms to gaseous pollutants and a corresponding in-situ toxicity detection system.

### BACKGROUND

Health risks caused by air pollution have always been one of the most urgent environmental problems all over the world. In 2021, the World Health Organization released the latest Global Air Quality Guidelines (AQG 2021), emphasizing the significant relationship between complex air pollution problems and a series of non-communicable diseases. These diseases include, but are not limited to, chronic respiratory system diseases, nervous system diseases, cardiovascular diseases, and immune system diseases. Therefore, it becomes imperative to quickly and economically learn about potential toxicity of the air environment where people live.

Air pollutants mainly include volatile organic compounds (VOCs) and atmospheric particulate matter that exist widely in various environments, and have profound impacts on human health and ecosystems. The air pollutants mainly come from industrial emission, traffic exhaust gas, building construction, agricultural activities, and natural processes. VOCs are usually released into air in a gaseous form due to volatility, whereas atmospheric particulate matter exists in the form of suspended particles. Both VOCs and atmospheric particulate matter can be inhaled into human body and cause harm to various organ systems. Fine atmospheric particulate matter (for example, PM2.5) can penetrate the shielding defense of human body due to small sizes, go deep into lungs, and enter the blood circulation system, causing oxidative stress and inflammatory reactions, and further harming the cardiovascular system. Some components such as benzene and formaldehyde of VOCs are considered having carcinogenic potentials, and long-term exposure to these components is closely related to increases in incidence rates of leukemia, lung cancer, and other organ tumors. In addition, VOCs and atmospheric particulate matter affect the functions of the central nervous system, liver, kidney, and other organs, and also weaken the immune system, making human body more vulnerable to infections, allergic reactions, and autoimmune diseases. At present, air quality management is mainly based on monitoring concentration of specific pollutants, for example, limit standards of VOCs and PM2.5. However, these methods are not sufficient to comprehensively reflect the complexity of air pollution. Air pollutants are usually formed of various mixed components, in which many chemical substances are very difficult to recognize, and pollutants in different districts have greatly varied components and toxicity effects. Therefore, pollution control solely depending on physical and chemical indicators has limitations. The assessment and management of toxicity effects based on in-situ exposure to air pollutants can reflect actual impacts of the air pollutants on human health more comprehensively and accurately.

At present, a mouse is used as a model in most researches of toxicity effects of direct exposure to air pollutants. This assessment manner is highly time consuming, and depends on complex and expensive analysis, for example, metagenomics and metabonomics. In addition, it is very difficult to eliminate impacts of individual differences of mice on results. Some other researchers construct a 3D cell model system to research toxicity effects of gaseous pollutants. However, such a technology still has some problems, including high costs, complex operations, a strict exposure compartment, and instable toxic feedbacks. Therefore, at present, the detection of toxicity effects of gaseous pollutants is still at the laboratory research stage, and cannot be applied to health effect-based air quality inspection and commercial detection. Some microorganisms having natural sensitivity to toxicity or some constructed recombined microorganisms having a gene pathway for specific toxic response has advantages of low costs, quick toxicity response, and stable toxicity feedback in toxicity detection. However, these microorganisms cannot be directly exposed to air, and have not been applied in toxicity detection of gaseous pollutants so far. In summary, there is still a lack of a low-cost, quick, in-situ toxicity assessment system that can cover various toxicity endpoints, and the research and development and the promotion of related technologies are urgently required, to cope with increasingly severe air pollution challenges.

### SUMMARY

In view of the shortcomings of the prior art described above, the present application aims to provide an exposure device for exposing microorganisms to gaseous pollutants, and also further provides a corresponding in-situ exposure toxicity detection system based on the exposure device. Specifically, the present application discloses a in-situ multi-toxicity endpoint detection system for VOCs and an in-situ multi-toxicity endpoint detection system for atmospheric particulate matter, to implement low-cost, quick, and high-flux detection of toxicity of gaseous pollutants; and provides in-situ multi-toxicity endpoint detection systems for VOCs and atmospheric particulate matter with low-cost, quick, multi-toxicity endpoint in-situ toxicity effect assessment.

An aspect of the present application provides an exposure device for exposing microorganisms to gaseous pollutants. The exposure device for exposing microorganisms to gaseous pollutants includes: a first depressurizing air inlet pipe, a second depressurizing air inlet pipe, a T-shaped air flow-mixing pipe, a multi-well exposure tray, and a pressurizing air outlet pipe.

Preferably, two depressurizing air inlet pipes are provided, one T-shaped air flow-mixing pipe is provided, and one multi-well exposure tray is provided; and the depressurizing air inlet pipes are configured to introduce gaseous pollutants, a structure of the first and second depressurizing air inlet pipes is funnel-shaped, a small end of the first and second depressurizing air inlet pipes is an air inlet, and a big end of the first and second depressurizing air inlet pipes is provided with threads; and diameters of the big ends of the first and second depressurizing air inlet pipes match with diameters of a left end and a right end of a horizontal pipe in the T-shaped air flow-mixing pipe, and the big ends of the first and second depressurizing air inlet pipes are respectively connected to the left end and the right end of the horizontal pipe in the T-shaped air flow-mixing pipe through the threads.

The multi-well exposure tray is arranged in a vertical pipe of the T-shaped air flow-mixing pipe, and is a cylinder in which a plurality of channels are uniformly distributed; four trapezoidal support pins are arranged in each channel, and are configured to support the hydrogel microspheres fixed with microorganisms, to make the hydrogel microspheres suspended in each channel; the plurality of channels are configured to provide a place for contact of the gaseous pollutants and the hydrogel microspheres; specifically, the gaseous pollutants continuously flow in the plurality of channels in the multi-well exposure tray through the first and second depressurizing air inlet pipes and are uniformly purged over surfaces of the hydrogel microspheres, to make the gaseous pollutants adhere to, accumulate at, and diffuse into the surfaces of the hydrogel microspheres, so that the microorganisms fixed in the hydrogel microspheres contact the gaseous pollutants. The hydrogel microspheres are provided with different types of microorganisms and are placed in the plurality of channels to form an array, so as to simultaneously detect multiple toxicity indicators.

In the present application, the first depressurizing air inlet pipe and the second depressurizing air inlet pipe have a structure of funnel to ensure that a velocity and a pressure of an airflow are reduced while a flow rate of the airflow is kept unchanged and to assist the T-shaped air flow-mixing pipe in uniformly distributing the airflow, so that the airflow enter from the left end and the right end of the horizontal pipe in the T-shaped air flow-mixing pipe impact each other to form a turbulence and change an airflow direction to ensure a same flow velocity and a same pressure at any position and in any cross-section of the vertical pipe of the T-shaped air flow-mixing pipe, as well as a consistent flow velocity and a consistent pressure when the airflow passing through each well in the multi-well exposure tray.

The pressurizing air outlet pipe is configured to discharge the gaseous pollutants, and is also funnel-shaped, a diameter of a big opening end of the pressurizing air outlet pipe matches with a diameter of the vertical pipe of the T-shaped air flow-mixing pipe, and the big opening end and the vertical pipe are connected through threads; the pressurizing air outlet pipe and the depressurizing air inlet pipe have opposite functions, the pressurizing air outlet pipe is located at a tail end of the vertical pipe of the T-shaped air flow-mixing pipe, and generates a particular pressure at a small opening end of the pressurizing air outlet pipe, so as to ensure uniformity and stability of the airflow after passing through the multi-well exposure tray.

Further, to fixedly connect the first and second depressurizing air inlet pipes and two ends of the T-shaped air flow-mixing pipe, the big ends of the first and second depressurizing air inlet pipes are respectively connected to the left end and the right end of the horizontal pipe in the T-shaped air flow-mixing pipe through the threads.

Further, a quantity of the channels in the multi-well exposure tray may range from 10 to 30, and a diameter of the channel ranges from 6 mm to 10 mm. The diameter of the hydrogel microspheres with microorganisms are smaller than the diameter of the channels, and for example, range from 4 mm to 8 mm.

Further, a plurality of multi-well exposure trays may be provided and are vertically connected through threads, and the multi-well exposure trays may be connected to each other to form a plurality of layers of exposure sets to multiply a quantity of the hydrogel microspheres placed in the multi-well exposure trays to expose microorganisms to the gaseous pollutants. For “the plurality”, a specific quantity may be determined according to an actual requirement, and specifically, for example, ranges from 2 to 4.

Preferably, in the embodiments of the present application, the diameters of the left end and the right end of the horizontal pipe in the T-shaped air flow-mixing pipe are 30 mm; the diameter of the small end of the first and second depressurizing air inlet pipes is 6 mm, and the diameter of the big end of the first and second depressurizing air inlet pipes 30 mm; the diameter of the vertical pipe of the T-shaped air flow-mixing pipe is 30 mm; and the diameter of the big end of the pressurizing air outlet pipe is 30 mm, and the diameter of the big end of the pressurizing air outlet pipe is 6 mm.

Further, preferably, in the embodiments of the present application, the quantity of the channels is 17, and the diameter of the channel is 8 mm. The diameters of the hydrogel microspheres with microorganisms are 6 mm.

Preferably, for the device in the present application, a main part of the device may be made of mirror-polished stainless steel.

A method for using the exposure device for exposing microorganisms to gaseous pollutants provided in the present application includes steps as follows:
(1) preparing the hydrogel microspheres with microorganisms: culturing microorganisms in a culture medium dissolved with 1.5% of sodium alginate for 3 h to 4 h, and after OD600 is greater than 0.1, uniformly dripping the microorganisms into a 1.5% calcium chloride solution through a peristaltic pump at a rate of 1.5 ml/min to obtain the hydrogel microspheres with microorganisms; taking out and placing the hydrogel microspheres in a culture medium for use;
(2) sequentially loading the hydrogel microspheres fixed with microorganisms in the multi-well exposure tray according to a detection requirement, and detecting an initial intensity of fluorescent signals of the hydrogel microspheres fixed with microorganisms;
(3) fixing the multi-well exposure tray to a lower end of the vertical pipe of the T-shaped air flow-mixing pipe through threads, and connecting the pressurizing air outlet pipe to the T-shaped air flow-mixing pipe, wherein silicone seal gaskets are mounted at all threaded connections to ensure leakproofness of the exposure device;
(4) placing the exposure device in an in-situ multi-toxicity endpoint detection system for VOCs or an in-situ multi-toxicity endpoint detection system for atmospheric particulate matter to make the gaseous pollutants enter the exposure device through the first and second depressurizing air inlet pipes at a flow rate ranging from 0.3 L/min to 2.0 L/min; and
(5) continuously introducing the gaseous pollutants for 15 min to 60 min, and then introducing clean air for 8 min to 10 min to completely replace the gaseous pollutants in the detection system; removing the multi-well exposure tray, and sequentially placing the hydrogel microspheres in the multi-well exposure tray into a 96 well plate, and detecting a final intensity of fluorescent signals of the hydrogel microspheres fixed with microorganisms in the plurality of channels by using a microplate reader to obtain an intensity change of fluorescent signals.

The first and second depressurizing air inlet pipes and the pressurizing air inlet pipe in the present application are both connected to other parts of the in-situ multi-toxicity endpoint detection system for VOCs or the in-situ multi-toxicity endpoint detection system for atmospheric particulate matter through metal pipes with a diameter of 10 mm.

The flow rate of the gaseous pollutants in the present application is preferably 0.5 L/min, and preferably does not exceed 2.0 L/min at most, to avoid rapid water loss of the hydrogel microspheres with microorganisms under the gaseous pollutants with a high flow velocity, thereby avoiding the death of the fixed microorganisms.

The shortest exposure time of the gaseous pollutants in the present application is preferably not shorter than 15 min, and the longest exposure time cannot exceed 60 min, and preferably ranges from 20 min to 30 min.

A second aspect of the present application provides an in-situ multi-toxicity endpoint detection system for VOCs. The in-situ multi-toxicity endpoint detection system for VOCs includes the exposure device for exposing microorganisms to gaseous pollutants.

Preferably, the in-situ multi-toxicity endpoint detection system for VOCs includes a concentration device, a distribution device, an exposure device, a gas circulation device, a tail gas treatment device, a detection device, and a cleaning device.

The concentration device comprises an atmospheric pre-concentrator and an atmospheric sampling vacuum pump that are connected, VOCs are collected and concentrated on site and introduced into the exposure device to perform toxicity effect detection; or to-be-tested air samples from a target site are collected by using a SUMMA canister, and then the SUMMA canister is loaded on the atmospheric pre-concentrator to concentrate VOCs to perform toxicity effect detection. A concentration ratio of VOCs is made stable and controllable in a manner of adsorption-thermal desorption in cooperation with a carrier gas with a precise and controllable flow rate. The concentration device is configured to collect VOCs in air, and concentrate and accumulate VOCs until a response threshold of the in-situ multi-toxicity endpoint detection system for VOCs is reached.

The distribution device is a borosilicate glass gas manifold, in which concentrated VOCs from the concentration device and VOCs from the gas circulation device are evenly mixed to form a mixture, and the mixture enters the exposure device with equal amounts through gas circuits at two ends of the exposure device.

The hydrogel microspheres fixed with microorganisms for different toxicity detection are arranged in the exposure device for exposing the microorganisms to the gaseous pollutants, and the microorganisms are in full contact with the concentrated VOCs with a highly stable concentration and a highly stable flow rate from the distribution device.

Preferably, the exposure device for exposing microorganisms to gaseous pollutants includes: a first depressurizing air inlet pipe, a second depressurizing air inlet pipe, a T-shaped air flow-mixing pipe, a multi-well exposure tray, and a pressurizing air outlet pipe.

The first depressurizing air inlet pipe, and the second depressurizing air inlet pipe are configured to introduce the gaseous pollutants (the VOCs), a structure of the first and second depressurizing air inlet pipes is funnel-shaped, a small end of the first and second depressurizing air inlet pipes is an air inlet, and a big end of the first and second depressurizing air inlet pipes is provided with threads; and diameters of the big ends of the first and second depressurizing air inlet pipes match with diameters of a left end and a right end of a horizontal pipe in the T-shaped air flow-mixing pipe, and the big ends of the first and second depressurizing air inlet pipes are respectively connected to the left end and the right end of the horizontal pipe in the T-shaped air flow-mixing pipe through the threads.

The multi-well exposure tray is arranged in a vertical pipe of the T-shaped air flow-mixing pipe, and is a cylinder in which a plurality of channels are uniformly distributed, four trapezoidal support pins are arranged in each channel, and are configured to support hydrogel microspheres fixed with microorganisms, to make the hydrogel microspheres suspended in each channel; the plurality of channels are configured to provide a place for contact of the gaseous pollutants (the VOCs) and the hydrogel microspheres; specifically, the gaseous pollutants (the VOCs) continuously flows in the plurality of channels in the multi-well exposure tray through the first and second depressurizing air inlet pipes and are uniformly purged over surfaces of the hydrogel microspheres to make the gaseous pollutants (the VOCs) adhere to, accumulate at, and diffuse into the surfaces of the hydrogel microspheres, so that the microorganisms fixed in the hydrogel microspheres contact the gaseous pollutants (the VOCs). The hydrogel microspheres are provided with different types of microorganisms and are placed in the plurality of channels to form an array, so as to simultaneously detecting multiple toxicity indicators.

In the present application, the first depressurizing air inlet pipe and the second depressurizing air inlet pipe have a structure of funnel to ensure that a velocity and a pressure of an airflow are reduced while a flow rate of the airflow is kept unchanged, and to assist the T-shaped air flow-mixing pipe in uniformly distributing the airflow, so that the airflow enter from left end and the right end of the horizontal pipe in the T-shaped air flow-mixing pipe impact each other in the middle of the T-shaped air flow-mixing pipe to form a turbulence and change an airflow direction to ensure a same flow velocity and a same pressure at any position and in any cross-section of the vertical pipe of the T-shaped air flow-mixing pipe, as well as a consistent flow velocity and a consistent pressure when the airflow passing through each well in the multi-well exposure tray.

The pressurizing air outlet pipe is configured to discharge the gaseous particulate pollutants, a structure of the pressurizing air outlet pipe is also funnel-shaped, a diameter of a big opening end of the pressurizing air outlet pipe matches with a diameter of the vertical pipe of the T-shaped air flow-mixing pipe, and the big opening end and the vertical pipe are connected through threads; the pressurizing air outlet pipe and the depressurizing air inlet pipe have opposite functions, the pressurizing air outlet pipe is located at a tail end of the vertical pipe of the T-shaped air flow-mixing pipe, and generates a particular pressure at a small opening end of the pressurizing air outlet pipe, so as to ensure uniformity and stability of the airflow after passing through the multi-well exposure tray.

The gas circulation device comprises a vacuum pump and a flowmeter that are connected, and is configured to circulate VOCs flowing out of the exposure device back to the distribution device to fully mix with the concentrated VOCs that newly enters the distribution device, thereby increasing concentration of VOCs and exposure efficiency to VOCs in the exposure device.

The tail gas treatment device is a multistage treatment system, where a primary treatment device is a gas-washing bottle that contains 50% of pure water and 50% of ethanol and is configured to absorb most VOCs that flows out of the exposure device; a secondary treatment device is a silica-gel drying tube, configured to remove moisture that a tail gas contains; a tertiary treatment device is two activated carbon absorption tubes connected end to end and is configured to completely capture remaining VOCs in the tail gas; and a system-controlled flowmeter and a system-controlled vacuum pump are arranged at a distal end of the detection system, and are configured to control and stabilize an airflow rate in the detection system.

To avoid secondary pollution caused by discharge of VOCs in the detection system to environment, a system-controlled flowmeter and a system-controlled vacuum pump are arranged at a distal end of the in-situ multi-toxicity endpoint detection system for VOCs, and are configured to control and stabilize an airflow rate in the detection system.

The multi-well exposure tray in the exposure device is designed as a detachable structure, so that quick assembly and disassembly can be performed.

The detection device is a microplate reader and is configured to detect changes in fluorescence intensity of the hydrogel microspheres fixed with the microorganisms before and after exposure.

The cleaning device is located at an upstream of the distribution device, is a high-temperature steam generator equipped with a high-purity air carrier gas, and generates high-temperature steam containing 50% of ethanol and 50% of pure water; the high-temperature steam is carried by the high-purity air carrier gas to purge the detection system after toxicity detection is completed.

In the in-situ multi-toxicity endpoint detection system for VOCs, wherein the exposure device, the concentration device, the distribution device, the gas circulation device, the tail gas treatment device, the detection device, and the cleaning device are connected through metal pipes with a diameter of 10 mm, the detection system is an absolutely sealed system, all connections of the detection system are sealed by silicone seal gaskets to ensure airtightness of the detection system, so as to avoid secondary pollution to environment and adverse health effects on testing personnel caused by leakage of concentrated VOCs.

A connection manner of the exposure device, the concentration device, the distribution device, the gas circulation device, the tail gas treatment device, the detection device, and the cleaning device in the detection system is as follows: three air inlets of the distribution device are sequentially connected to the atmospheric pre-concentrator, a distal end of the gas circulation device, and the high-temperature steam generator; two air outlets of the gas distribution device are respectively connected to the first and second depressurizing air inlet pipes of the exposure device; the pressurizing outlet pipe of the exposure device is connected to a first end of a three-way pipe, a second end of the three-way pipe is connected to the flowmeter of the gas circulation device which is then connected to the vacuum pump of the gas circulation device to circulate VOCs to the distribution device, and a third end of the three-way pipe is connected to the gas-washing bottle of the tail gas treatment device; the gas-washing bottle, the silica-gel drying tube, the two activated carbon adsorption tubes, the system-controlled flowmeter, and the system-controlled vacuum pump are sequentially connected. The microplate reader for detection is placed next to the exposure device and is not directly connected to the system.

A working procedure of the in-situ multi-toxicity endpoint detection system for VOCs of the present application is as follows:
(1) selecting an appropriate adsorption material according to physical and chemical properties of VOCs contained in to-be-tested air samples, filling the adsorption material in an adsorber of the atmospheric pre-concentrator, and adding a mixed liquid of 50% of ethanol and 50% of pure water to the gas-washing bottle and the high-temperature steam generator;
(2) determining, according to a detection requirement, a quantity of the multi-well exposure trays that need to be loaded and a type and a quantity of the hydrogel microspheres that need to be used; detecting and recording an initial fluorescence intensity of each hydrogel microsphere; placing the hydrogel microspheres in the multi-well exposure trays, loading the multi-well exposure tray into the exposure device, and sealing the detection system;
(3) introducing air to allow the air to pass through the detection system at 2 L/min, and determining whether values displayed by flowmeters of the detection system are the same and stable, thereby ensuring the airtightness of the detection system;
(4) determining the concentration ratio of VOCs, and turning on the atmospheric sampling vacuum pump to collect VOCs in air of a target site at a particular flow rate, or connecting the SUMMA canister to the atmospheric pre-concentrator to concentrate VOCs in a pre-collected air sample; after adsorption of VOCs is completed, increasing a temperature of the adsorber to 200° C. to 600° C. to make VOCs adsorbed and concentrated in the adsorption material into a gaseous state, wherein a thermal desorption time ranges from 15 min to 60 min; mixing air and VOCs in the gaseous state; and obtaining a mixed gas containing VOCs with an accurate and controllable concentration ratio by controlling a rate of thermal desorption rate and a flow rate of the carrier gas;
(5) turning on the system-controlled vacuum pump at the distal end of the detection system and the vacuum pump of the gas circulation device to introduce the concentrated VOCs into the exposure device at 0.5 L/min to 2.0 L/min, wherein exposure lasts 15 min to 60 min; and monitoring stability of the flowmeter of the gas circulation device and the system-controlled flowmeter at the distal end of the detection system during the process;
(6) after exposure to VOCs is completed, introducing pure air into the detection system for 8 min to 12 min, taking out the multi-well exposure tray from the exposure device and transferring the hydrogel microspheres from the multi-well exposure trays into a 96 well plate, and detecting final fluorescence intensity of each hydrogel microsphere by using the microplate reader; comparing and calculating changes of the initial fluorescence intensity and the final fluorescence intensity to obtain toxicity effect data of this exposure; and
(7) after exposure to VOCs is completed, performing ultrasonic cleaning on the multi-well exposure tray for 15 min to 30 min by using the mixed liquid of 50% of pure water and 50% of ethanol, then loading the multi-well exposure tray back to the exposure device, and sealing, and checking the airtightness of the detection system; turning on the high-temperature steam generator, wherein a temperature of the high-temperature steam generator is set to 110° C. to 130° C., injecting the mixed liquid of 50% of pure water and 50% of ethanol into a vaporizer of the high-temperature steam generator through a precision injection pump at 2 L/min to 4 L/min, and carrying the mixed liquid to enter the detection system through high-purity air at 2 L/min to 4 L/min to perform cleaning for 10 min to 20 min; and then turning off the high-temperature steam generator, and purging the detection system with high-purity air at 2 L/min to 4 L/min for 5 min to 10 min.

A third aspect of the present application provides an in-situ multi-toxicity endpoint detection system for atmospheric particulate matter. The in-situ multi-toxicity endpoint detection system for atmospheric particulate matter includes the exposure device for exposing microorganisms to gaseous pollutants of the present application.

Preferably, the in-situ multi-toxicity endpoint detection system for atmospheric particulate matter includes an atmospheric particulate matter concentration and accumulation device, an atmospheric particulate matter concentration control device, a gas distribution device, a exposure device, a circulation device, and a tail gas treatment device.

The atmospheric particulate matter concentration and accumulation device includes an atmospheric sampling pump, an atmospheric particulate matter sampler, a heating water tank, a condensation circulation device, and a virtual impaction head.

The atmospheric particulate matter sampler is provided with three impaction heads of: PM2.5, PM5, and PM10, to collect atmospheric particulate matter with different particle sizes in air. Under an action of a suction force of the atmospheric sampling pump, air samples are collected, and after being sorted by the three impaction heads, the atmospheric particulate matter of particular particle sizes enters the heating water tank; and a heating rod is arranged in the heating water tank to heat deionized water to 40 degrees Celsius to 50 degrees Celsius to generate sufficient water vapor to obtain atmospheric particulate matter in a saturated state.

The atmospheric particulate matter in a saturated state enters the condensation circulation device at a flow rate of 30 L/min to 50 L/min in a main gas circuit, and a temperature in the condensation circulation device ranges from −18 degrees Celsius to 25 degrees Celsius, so that the atmospheric particulate matter in a saturated state gradually condense and grow large in a condensation tube to eventually form large atmospheric particulate matter with particle diameters ranging from 3 micrometers to 4 micrometers; and eventually the large atmospheric particulate matter passes through the virtual impaction head at a flow rate of 1 L/min to 6 L/min in a concentration gas circuit, exits from the atmospheric particulate matter concentration and accumulation device at a nozzle at an increased speed, and enters the atmospheric particulate matter concentration control device.

The atmospheric particulate matter concentration control device provides a closed space and comprises an atmospheric particulate matter concentration laser detector and a high-purity compressed air cylinder; the atmospheric particulate matter concentration laser detector is configured to continuously monitor a concentration of the atmospheric particulate matter that flows from the atmospheric particulate matter concentration and accumulation device; and it is determined, according to a concentration value of the atmospheric particulate matter, whether to increase an accumulation ratio of the atmospheric particulate matter concentration and accumulation device, or to open the high-purity compressed air cylinder, to precisely control a flow rate of a high-purity air carrier gas that is introduced into the atmospheric particulate matter concentration control device; mixing of the high-purity air carrier gas with the atmospheric particulate matter is performed to obtain a mixed gas, thereby implementing precise control of a concentration of the atmospheric particulate matter, and determining a conversion relationship between the concentration of the atmospheric particulate matter that the multi-well exposure tray is exposed to and an actual concentration of the atmospheric particulate matter in air.

The gas distribution device is configured to distribute the mixed gas containing the atmospheric particulate matter with an accurate concentration from the atmospheric particulate matter concentration control device, fully and uniformly mix the mixed gas with the atmospheric particulate matter circulated back from the circulation device to obtain a mixture, and the enable mixture of the mixed gas and the atmospheric particulate matter circulated back from the circulation device to enter the exposure device with equal amounts through the first and second depressurizing air inlet pipe; and specifically, the gas distribution device is a borosilicate glass gas distribution tube.

The hydrogel microspheres fixed with microorganisms for different toxicity detection are arranged in the exposure device for exposing the microorganisms to the gaseous pollutants, and are in full contact with the mixture with a highly stable concentration and a highly stable flow rate from the gas distribution device, to expose the microorganisms to the atmospheric particulate matter.

Preferably, the exposure device for exposing microorganisms to gaseous pollutants includes: a first depressurizing air inlet pipe, a second depressurizing air inlet pipe, a T-shaped air flow-mixing pipe, a multi-well exposure tray, and a pressurizing air outlet pipe.

The first depressurizing air inlet pipe, and the second depressurizing air inlet pipe are configured to introduce atmospheric particulate matter, a structure of the first and second depressurizing air inlet pipes is funnel-shaped, a small end of the first and second depressurizing air inlet pipes is an air inlet, and a big end of the first and second depressurizing air inlet pipes is provided with threads; and diameters of the big ends of the first and second depressurizing air inlet pipes match with diameters of a left end and a right end of a horizontal pipe in the T-shaped air flow-mixing pipe, and the big ends of the first and second depressurizing air inlet pipes are respectively connected to the left end and the right end of the horizontal pipe in the T-shaped air flow-mixing pipe through the threads.

The multi-well exposure tray is arranged in a vertical pipe of the T-shaped air flow-mixing pipe, and is a cylinder in which a plurality of channels are uniformly distributed, four trapezoidal support pins are arranged in each channel, and are configured to support hydrogel microspheres fixed with microorganisms, to make the hydrogel microspheres suspended in each channel; the plurality of channels are configured to provide a place for contact of the atmospheric particulate matter and the hydrogel microspheres; specifically, the atmospheric particulate matter continuously flow in the plurality of channels in the multi-well exposure tray through the first and second depressurizing air inlet pipes and are uniformly purged over surfaces of the hydrogel microspheres to make the atmospheric particulate matter adhere to, accumulate at, and diffuse into the surfaces of the hydrogel microspheres, so that the microorganisms fixed in the hydrogel microspheres contact the atmospheric particulate matter. The hydrogel microspheres are provided with different types of microorganisms and are placed in the plurality of channels to form an array, so as to simultaneously detecting multiple toxicity indicators.

In the present application, the first depressurizing air inlet pipe and the second depressurizing air inlet pipe have a structure of funnel to ensure that a velocity and a pressure of an airflow are reduced while a flow rate of the airflow is kept unchanged, and to assist the T-shaped air flow-mixing pipe in uniformly distributing the airflow, so that the airflow enter from left end and the right end of the horizontal pipe in the T-shaped air flow-mixing pipe impact each other in the middle of the T-shaped air flow-mixing pipe to form a turbulence and change an airflow direction to ensure a same flow velocity and a same pressure at any position and in any cross-section of the vertical pipe of the T-shaped air flow-mixing pipe, as well as a consistent flow velocity and a consistent pressure when the airflow passing through each well in the multi-well exposure tray.

The pressurizing air outlet pipe is configured to discharge the gaseous particulate pollutants, a structure of the pressurizing air outlet pipe is also funnel-shaped, a diameter of a big opening end of the pressurizing air outlet pipe matches with a diameter of the vertical pipe of the T-shaped air flow-mixing pipe, and the big opening end and the vertical pipe are connected through threads; the pressurizing air outlet pipe and the depressurizing air inlet pipe have opposite functions, the pressurizing air outlet pipe is located at a tail end of the vertical pipe of the T-shaped air flow-mixing pipe, and generates a particular pressure at a small opening end of the pressurizing air outlet pipe, so as to ensure uniformity and stability of the airflow after passing through the multi-well exposure tray.

The multi-well exposure tray in the exposure device is designed as a detachable structure, so that quick assembly and disassembly can be implemented.

The circulation device comprises a tubular axial-flow fan and an electronic soap film flowmeter, the concentrated atmospheric particulate matter that flows out of the exposure device is circulated back to the gas distribution device and is fully mixed with the mixed gas containing the atmospheric particulate matter that enters the gas distribution device to increase a concentration of the atmospheric particulate matter and exposure efficiency to the of atmospheric particulate matter in the exposure device.

The tail gas treatment device is a multistage treatment system; wherein a primary treatment device is a silica-gel drying tube, configured to adsorb moisture absorbed by the atmospheric particulate matter in a concentration and accumulation process; a secondary treatment device and a tertiary treatment device are two atmospheric particulate matter filters that are connected end to end, and are configured to avoid secondary pollution caused by discharge of the atmospheric particulate matter in the detection system to environment; and a system-controlled flowmeter and a system-controlled vacuum pump are arranged at a distal end of the detection system, and are configured to control and stabilize an airflow rate in the detection system.

Preferably, the in-situ multi-toxicity endpoint detection system for atmospheric particulate matter of the present application further includes a detector, and the detector is a microplate reader, and is not connected to other parts of the detection system. After exposure of the multi-well exposure tray to the atmospheric particulate matter is completed, the multi-well exposure tray in the exposure device is quickly disassembled, the hydrogel microspheres fixed with microorganisms in the multi-well exposure tray are placed into a 96 well plate, and each hydrogel microsphere in the 96 well plate is subjected to the microplate reader for fluorescence intensity detection.

Preferably, the in-situ multi-toxicity endpoint detection system for atmospheric particulate matter of the present application further includes a cleaning device, and the cleaning device is located upstream of the gas distribution device, and comprises a high-temperature steam generator equipped with a pure air carrier gas; after exposure of the multi-well exposure tray to the atmospheric particulate matter is completed, the exposure device is easily disassembled using a quick-connect buckle provided on the exposure device. The exposure device then is placed in an ultrasonic cleaning machine that contains 50% of ethanol and 50% of pure water to perform ultrasonic cleaning for 15 min to 30 min, followed by loading back into the exposure device, sealing, and airtightness checking; the high-temperature steam generator is turned on, wherein a temperature of the high-temperature steam generator is set to 110° C. to 130° C., a mixed liquid of 50% of pure water and 50% of ethanol is injected into a vaporizer through a precision injection pump at 2 L/min to 4 L/min, and the mixed liquid is carried to enter the detection system through high-purity air at 2 L/min to 4 L/min to perform cleaning for 10 min to 20 min; and then the high-temperature steam generator is turned off, and the system is purged with air at 2 L/min to 4 L/min for 5 min to 10 min.

Preferably, in the in-situ multi-toxicity endpoint detection system for atmospheric particulate matter of the present application, the atmospheric particulate matter concentration and accumulation device, the atmospheric particulate matter concentration control device, the gas distribution device, the circulation device, and the tail gas treatment device in the detection system are connected through metal pipes with a diameter of 10 mm, the detection system is an absolutely sealed system, and all connection are sealed by silicone seal gaskets to ensure airtightness of the detection system, so as to avoid secondary pollution to environment and adverse health effects on testing personnel caused by leakage of concentrated atmospheric particulate matter.

A connection manner in the detection system of the present application is as follows:
the atmospheric particulate matter concentration and accumulation device is connected to one air inlet of the atmospheric particulate matter concentration control device, a second air inlet of the atmospheric particulate matter concentration control device is connected to the high-purity compressed air cylinder, and a probe of the atmospheric particulate matter concentration laser detector is fixed on an inner wall of the atmospheric particulate matter concentration control device; an outlet of the atmospheric particulate matter concentration control device is connected to a first air inlet of the gas distribution device; a second and a third air inlets of the gas distribution device are respectively connected to a distal end of the circulation device and the high-temperature steam generator; a first and a second air outlets of the gas distribution device are connected to the first and second depressurizing air inlet pipes of the exposure device through the quick-connect buckles, the pressurizing air outlet pipe of the exposure device is connected to a three-way pipe through the quick-connect buckle, and one remaining end of the three-way pipe is connected to the electronic soap film flowmeter of the circulation device that is connected to the tubular axial-flow fan, wherein the tubular axial-flow fan is connected to the gas distribution device; the pressurizing air outlet pipe of the exposure device is also connected to the silica-gel drying tube of the tail gas treatment device; and the silica-gel drying tube, the atmospheric particulate matter filters, the system-controlled flowmeter, and the system-controlled vacuum pump are sequentially connected.

A working procedure of the in-situ multi-toxicity endpoint detection system for atmospheric particulate matter of the present application is as follows:
(1) adding deionized water to the heating water tank of the atmospheric particulate matter concentration and accumulation device to two-thirds of the heating water tank; adding ethanol to the condensation circulation device of the atmospheric particulate matter concentration and accumulation device, and adding a mixed liquid of 50% of ethanol and 50% of pure water to the high-temperature steam generator;
(2) determining, according to a detection requirement, a quantity of the multi-well exposure trays that need to be loaded and a type and a quantity of the hydrogel microspheres fixed with microorganism that need to be used; detecting and recording an initial fluorescence intensity of each hydrogel microsphere, placing the hydrogel microspheres in the multi-well exposure tray, and loading the multi-well exposure trays into the exposure device, and sealing the detection system;
(3) introducing high-purity air to allow the high-purity air to pass through the detection system at 2 L/min, and detecting whether values displayed by flowmeters of the detection system are the same and stable, thereby ensuring the airtightness of the detection system;
(4) determining a concentration ratio of the atmospheric particulate matter, turning on the atmospheric sampling pump, and collecting the atmospheric particulate matter in air of a target site; after the atmospheric particulate matter enter the atmospheric particulate matter concentration and accumulation device, starting a concentration procedure; and continuously monitoring changes in a concentration of the atmospheric particulate matter concentration in the atmospheric particulate matter concentration control device, and adjusting the accumulation ratio of the atmospheric particulate matter concentration and accumulation device, or introducing air with precise flow rate to mix with the atmospheric particulate matter to obtain the mixed gas, until the concentration of the atmospheric particulate matter in the atmospheric particulate matter concentration control device meets the detection requirement;
(5) turning on the system-controlled vacuum pump at the distal end of the detection system and the tubular axial-flow fan of the circulation device, and introducing the mixed gas into the exposure device at 0.5 L/min to 2.0 L/min, where exposure lasts 15 min to 60 min; and continuously monitoring the concentration of the atmospheric particulate matter in the atmospheric particulate matter concentration control device and stability of the electronic soap film flowmeter of the circulation device and the system-controlled flowmeter at the distal end of the detection system during the process;
(6) after exposure to the atmospheric particulate matter is completed, turning off the atmospheric particulate matter concentration and accumulation device and the atmospheric particulate matter concentration control device, introducing pure air into the detection system for 10 min to 20 min, taking out the multi-well exposure tray from the exposure device; and transferring the hydrogel microspheres fixed with microorganisms from in the multi-well exposure tray into a 96 well plate, and detecting final fluorescence intensity of the hydrogel microspheres fixed with microorganisms by using the microplate reader; comparing and calculating changes of the initial fluorescence intensity and the final fluorescence intensity to obtain toxicity effect data of this current exposure; and
(7) after exposure to the atmospheric particulate matter is completed, performing ultrasonic cleaning on the multi-well exposure trays for 15 min to 30 min by using the mixed liquid of 50% of pure water and 50% of ethanol, then loading the multi-well exposure tray back to the exposure device, and sealing and checking airtightness of the detection system; turning on the high-temperature steam generator, wherein a temperature of the high-temperature steam generator is set to 110° C. to 130° C., injecting the mixed liquid of 50% of pure water and 50% of ethanol into the vaporizer through the precision injection pump at 2 L/min to 4 L/min, and carrying the mixed liquid to enter the detection system through the high-purity air at 2 L/min to 4 L/min to perform cleaning for 10 min to 20 min; and then turning off the high-temperature steam generator, and purging the detection system with the high-purity air at 2 L/min to 4 L/min for 5 min to 10 min.

The beneficial effects of the present application are as follows:

An objective of the present application is to provide a method for quickly fixing microorganisms having natural sensitivity or recombined microorganisms having a gene pathway for specific toxic response based on calcium alginate hydrogel into hydrogel microspheres, assembling the hydrogel microspheres into a multi-well exposure tray, directly exposing microorganisms to air, and uniformly and continuously blowing concentrated air pollutants to the hydrogel microspheres with microorganisms to make air pollutants adhere to, accumulate at, and diffuse into surfaces of the hydrogel microspheres with microorganisms, to induce toxic response of microorganisms. Based on this, the present application provides an exposure device for exposing microorganisms to gaseous pollutants, and also further provides an air pollutant in-situ multi-toxicity endpoint exposure toxicity detection system based on the exposure device. Specifically, as an example, the present application separately provides an in-situ multi-toxicity endpoint detection system for VOCs and an in-situ multi-toxicity endpoint detection system for atmospheric particulate matter. Each of the in-situ multi-toxicity endpoint detection system for VOCs and the in-situ multi-toxicity endpoint detection system for atmospheric particulate matter includes the exposure device for exposing microorganisms to gaseous pollutants.

The detection system constructed in the present application may perform controlled accumulation of pollutants in air, and implements in-situ, comprehensive, quick, and low-cost assessment of toxicity effects of VOCs in air of a target site. The in-situ toxicity assessment can assess toxicity effects of air pollutants in the environment more genuinely and more comprehensively. Toxicity distortion caused by a complex collection and transfer process and complex chemical reactions in a liquid elution process of gaseous pollutants in such conventional gaseous pollutant toxicity detection through sampling, elution and collection, and liquid exposure is effectively avoided. Tens or even hundreds of hydrogel microspheres fixed with microorganisms that can detect acute toxicity, oxidative stress, DNA damage, protein damage, cell function damage, and the like can be flexibly assembled on a plurality of layers of multi-well exposure tray according to detection requirements, to implement customized detection of multi-toxicity endpoints of gaseous pollutants, thereby providing the system with high expandability and flexibility. The system of the present application has high stability and reproducibility in a real exposure process. In addition, changes in a detection environment cause no significant impact to the stability of the system. Through an integrated design, the system can be used for in-situ monitoring of toxicity effects of emission sources of priority gaseous pollutants, and can also be used for assessing toxicity effects when the concentration of urban atmospheric particulate matter rises.

### BRIEF DESCRIPTION OF THE DRAWINGS

FIG. 1 is a schematic structural diagram of an exposure device for exposing microorganisms to gaseous pollutants according to the present application.

FIG. 2 is a schematic structural diagram of a multi-well exposure tray of an exposure device for exposing microorganisms to gaseous pollutants, where (a) is an overall schematic structural diagram, (b) is a front view of the multi-well exposure tray, and (c) is a top view of the multi-well exposure tray.

FIG. 3 is a schematic structural diagram of a volatile organic compounds in-situ multi-toxicity endpoint exposure toxicity detection system.

FIG. 4 is a heat map of genotoxicity of dimethyl sulfate in in-situ determination using a in-situ multi-toxicity endpoint detection system for VOCs.

FIG. 5 is a concentration-acute toxicity curve of gaseous formaldehyde and gaseous phenol in in-situ determination using a in-situ multi-toxicity endpoint detection system for VOCs and shows comparison with a conventional method, where (a) is gaseous formaldehyde, (b) is liquid formaldehyde, (c) is gaseous phenol, and (d) is liquid phenol.

FIG. 6 is a schematic structural diagram of an in-situ multi-toxicity endpoint detection system for atmospheric particulate matter.

FIG. 7 is a schematic structural diagram of an atmospheric particulate matter concentration and accumulation device according to the present application.

FIG. 8 is a heat map of genotoxicity of atmospheric particulate matter from combustion of pine in in-situ determination using an in-situ multi-toxicity endpoint detection system for atmospheric particulate matter.

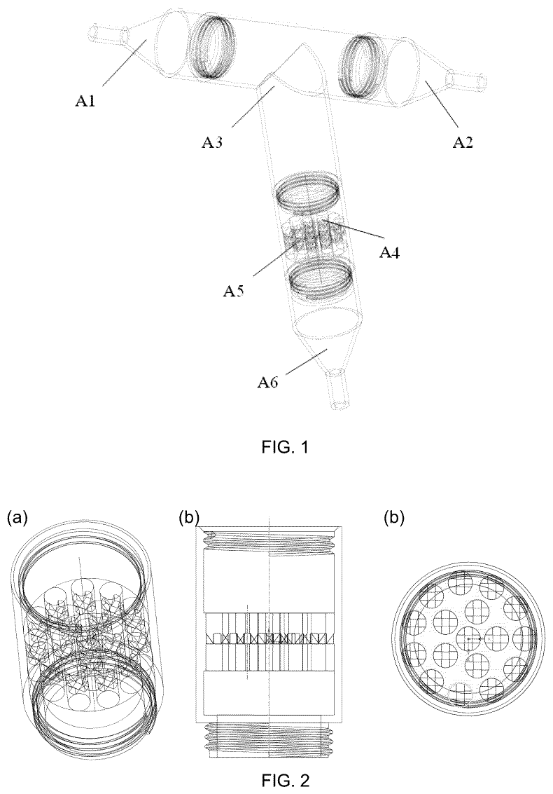

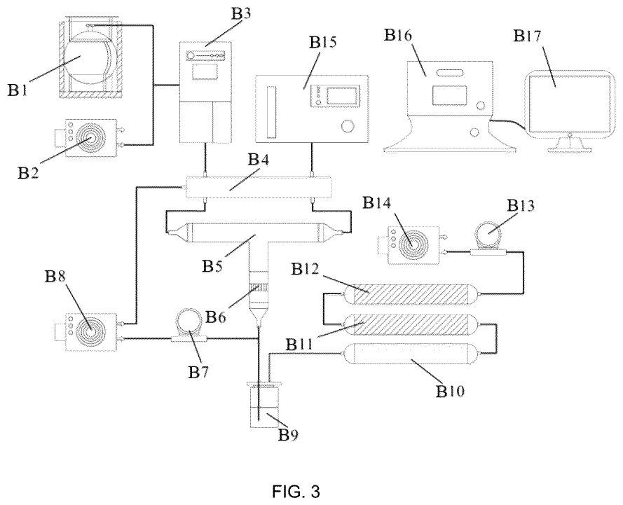

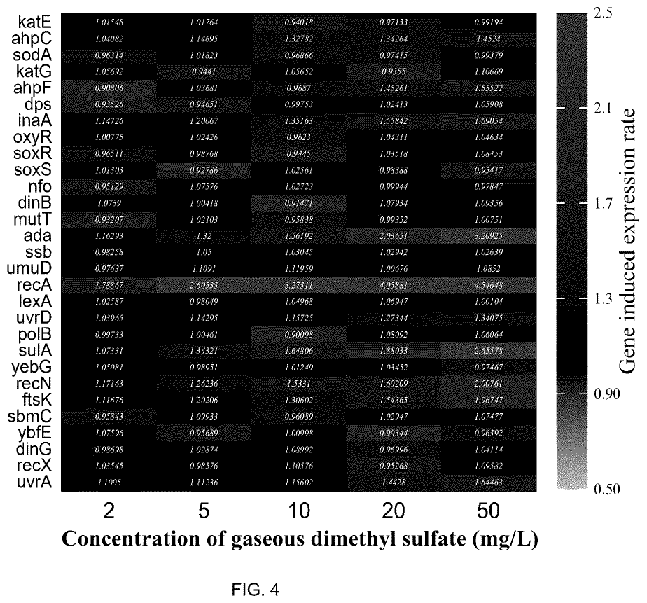

### REFERENCE NUMERALS

A1 First Depressurizing Air Inlet Pipe
A2 Second Depressurizing Air Inlet Pipe
A3 T-Shaped Air Flow-Mixing Pipe
A4 Multi-Well Exposure Tray
A5 Trapezoidal Support Pin
A6 Pressurizing Air Outlet Pipe
B1 SUMMA Canister
B2 Atmospheric Sampling Vacuum Pump
B3 Atmospheric Pre-concentrator
B4 Distribution Device
B5 Exposure Device
B6 Multi-Well Exposure Tray
B7 Flowmeter Of The Gas Circulation Device
B8 Vacuum Pump Of The Gas Circulation Device
B9 Primary Treatment Device (Gas-Washing Bottle)
B10 Secondary Treatment Device (Silica-Gel Drying Tube)
B11 Primary Activated Carbon Adsorption Tube
B12 Secondary Activated Carbon Adsorption Tube
B13 System-Controlled Flowmeter
B14 System-Controlled Vacuum Pump
B15 High-Temperature Steam Generator
B16 Microplate Reader
B17 Microplate Reader Computer
C1 Atmospheric Sampling Pump
C2 Atmospheric Particulate Matter Concentration and Accumulation Device
C3 Atmospheric Particulate Matter Concentration Laser Detector
C4 Atmospheric Particulate Matter Concentration Control Device
C5 High-purity Compressed Air Cylinder
C6 Gas Distribution Device
C7 Quick-Connect Buckle
C8 Exposure Device
C9 Multi-well Exposure Tray
C10 Electronic Soap Film Flowmeter Of The Circulation Device
C11 Tubular Axial-Flow Fan Of The Circulation Device
C12 Silica-Gel Drying Tube
C13 Primary Atmospheric Particulate Matter Filter
C14 Secondary Atmospheric Particulate Matter Filter
C15 System-Controlled Flowmeter
C16 System-Controlled Vacuum Pump
C17 High-Temperature Steam Generator
C18 Microplate Reader
C19 Microplate Reader Computer
C20 Atmospheric Particulate Matter Sampler
C21 Heating Water Tank Of The Atmospheric Particulate Matter Concentration and Accumulation Device
C22 Heating Rod In The Heating Water Tank
C23 Condensation Circulation Device
C24 Condensation Tube In The Condensation Circulation Device
C25 Vacuum Pump And Control Flowmeter In The Main Gas Circuit
C26 Vacuum Pump And Control Flowmeter In The Concentration Gas Circuit
C27 Virtual Impaction Head
C28 Nozzle

### DETAILED DESCRIPTION

The present application is further described below through the embodiments and the drawings.

### Part One: Exposure Device for Exposing Microorganisms to Gaseous Pollutants

A commercial VOCs generator is used to accurately generate seven different VOCs with nine gradient concentrations, involving aldehydes (formaldehyde and acetaldehyde), pyridines (pyridine), alcohols (n-butyl alcohol), acyls and ketones (acetylacetone), benzenes, and phenols (phenol). Natural luminescent bacteria Vibrio fischeri as a target, is fixed by using hydrogel microspheres, and is arranged in an exposure chamber. VOCs are sequentially introduced into the exposure chamber at a flow rate of 0.5 L/min. After exposure for 15 min, 30 min, 45 min, and 60 min, Vibrio fischeri is taken out, and intensity change of fluorescent signals of Vibrio fischeri are measured by using a microplate reader. Concentration-acute toxicity curves of the gaseous pollutant comprising different VOCs and mixed VOCs are fitted by using analysis software, to obtain the IC50 value of acute toxicity of the gaseous pollutants. Through the foregoing procedures and operations, acute toxicity detection of VOCs is implemented. Compared with corresponding acute toxicity exposure experiments of VOCs in a liquid state, the response (an IC50 value) of exposing Vibrio fischeri to gaseous pollutants is increased by 565.2 times to 14261.5 times. Common VOCs of formaldehyde and phenol are seen the most significant increases, a minimum detection limit of formaldehyde is 1.3 mg/m³, a minimum detection limit of phenol is 20.31 mg/m³, and a minimum detection limit of the mixed VOCs is 13.21 mg/m³, which are close to upper limits of air quality standards. In a toxicity detection process of gaseous pollutants in actual air, toxicity detection of trace gaseous pollutants can be easily implemented by using an automatic atmospheric pre-concentrator in cooperation. See Table 1 for details.

Table 1 shows sensitivity differences in acute toxicity of exposing to six types of VOCs in a gaseous state and a liquid state.

TABLE 1

| | IC50 (15 min) | | | IC50 (30 min) | | | IC50 (45 min) | | | IC50 (60 min) | | |
|---|---|---|---|---|---|---|---|---|---|---|---|---|
| VOCs | Gaseous | Liquid | Ratio | Gaseous | Liquid | Ratio | Gaseous | Liquid | Ratio | Gaseous | Liquid | Ratio |
| Formaldehyde | 4.81 | 16.33 | 3395 | 2.34 | 15.64 | 6684 | 1.81 | 15.42 | 8519 | 1.29 | 14.78 | 11457 |
| | μg/L | mg/L | | μg/L | mg/L | | μg/L | mg/L | | μg/L | mg/L | |
| Acetaldehyde | 2.12 | 0.64 | 302 | 1.29 | 0.58 | 450 | 0.83 | 0.58 | 699 | 0.49 | 0.60 | 1224 |
| | mg/L | g/L | | mg/L | g/L | | mg/L | g/L | | mg/L | g/L | |
| Pyridine | 1.50 | 1.02 | 680 | 1.25 | 1.08 | 864 | 0.88 | 1.14 | 1295 | 0.50 | 1.25 | 2500 |
| | mg/L | g/L | | mg/L | g/L | | mg/L | g/L | | mg/L | g/L | |
| N-butyl alcohol | 1.05 | 1.79 | 1705 | 0.73 | 1.84 | 2521 | 0.53 | 1.84 | 3472 | 0.40 | 1.85 | 4625 |
| | mg/L | g/L | | mg/L | g/L | | mg/L | g/L | | mg/L | g/L | |
| Acetylacetone | 0.28 | 0.28 | 1000 | 0.19 | 0.25 | 1316 | 0.13 | 0.31 | 2385 | 0.11 | 0.25 | 2273 |
| | mg/L | g/L | | mg/L | g/L | | mg/L | g/L | | mg/L | g/L | |
| Phenol | 68.86 | 0.27 | 3921 | 36.29 | 0.29 | 7991 | 28.01 | 0.32 | 11424 | 20.23 | 0.32 | 15818 |
| | μg/L | g/L | | μg/L | g/L | | μg/L | g/L | | μg/L | g/L | |
| Mixed VOCs | 33.29 | 0.19 | 5707 | 22.35 | 0.19 | 8501 | 16.46 | 0.17 | 10328 | 13.21 | 0.15 | 11355 |
| | μg/L | g/L | | μg/L | g/L | | μg/L | g/L | | μg/L | g/L | |

### Part Two: In-Situ Multi-Toxicity Endpoint Detection System for VOCs

Embodiment 1: Genotoxicity of an artificial gaseous pollutant (dimethyl sulfate) is detected by using the in-situ multi-toxicity endpoint detection system for volatile organic compounds (VOCs) constructed in the present application. Specific steps are as follows:

(1) It is ensured that all devices are in a good working state, and all necessary equipment and pipes are connected. In this embodiment, a Tenax TA multi-well polymer adsorbent is selected as the adsorption material to be filled in an adsorber of the atmospheric pre-concentrator, and a mixed liquid of 50% of ethanol and 50% of pure water is added to the gas-washing bottle and the high-temperature steam generator.

(2) According to a detection requirement in this embodiment, the quantity of the multi-well exposure tray that need to be loaded is 6, 29 types of hydrogel microspheres fixed with recombined Escherichia coli carrying plasmids with different gene pathway are provided, and the quantity of each type of hydrogel microspheres fixed with microorganisms that need to be used is 15. An initial fluorescence intensity of each hydrogel microsphere is detected and recorded, and the hydrogel microspheres are placed in the multi-well exposure tray, the multi-well exposure tray is loaded into the exposure device, and the detection system is sealed.

(3) Pure air is introduced to allow the pure air to pass through the detection system at 2 L/min, and it is determined whether values displayed by flowmeters of the detection system are the same and stable, thereby ensuring the airtightness of the detection system.

(4) Gaseous dimethyl sulfate with concentrations of 2 mg/m³, 5 mg/m³, 10 mg/m³, 20 mg/m³, and 50 mg/m³ are manually and continuously manufactured in a sealed space of 1 m³ by using a VOCs generator.

(5) It is determined that the concentration ratio of gaseous dimethyl sulfate is 10 times, the atmospheric sampling vacuum pump is turned on to collect gaseous dimethyl sulfate in the sealed space at a particular flow rate, and adsorption is performed in an automatic atmospheric pre-concentrator. After adsorption of VOCs is completed, a temperature of the adsorber is increased to 300° C. to make VOCs adsorbed and concentrated in the adsorption material into a gaseous state, wherein a thermal desorption rate is 10 mg/min, and a thermal desorption time is 15 min. The pure air is mixed with the concentrated gaseous dimethyl sulfate at a flow rate of 0.5 L/min, concentrations of the concentrated gaseous dimethyl sulfate entering the distribution device are sequentially 20 mg/m³, 50 mg/m³, 100 mg/m³, 200 mg/m³, and 500 mg/m³.

(6) The system-controlled vacuum pump at the distal end of the detection system and the vacuum pump of the gas circulation device are turned on, and the concentrated VOCs is introduced into the exposure device at 0.5 L/min, wherein exposure lasts 15 min; and stability of the flowmeter of the gas circulation device and the system-controlled flowmeter at the distal end of the detection system during the process is monitored.

(7) After exposure to VOCs is completed, pure air is introduced into the detection system for 10 min, the exposure device is turned on, and the multi-well exposure tray is taken out from the exposure device; the hydrogel microspheres from the multi-well exposure tray are sequentially transferred into a 96 well plate, and the hydrogel microspheres are placed into the microplate reader to detect final fluorescence intensity of each hydrogel microsphere by using the microplate reader; changes of the initial fluorescence intensity and the final fluorescence intensity are compared and calculated to obtain toxicity effect data of this exposure.

(8) After exposure to VOCs is completed, ultrasonic cleaning is performed on the multi-well exposure tray for 15 min by using the mixed liquid of 50% of pure water and 50% of ethanol, the multi-well exposure tray is loaded back to the exposure device, sealing the exposure device is performed, and the airtightness of the detection system is checked; the high-temperature steam generator is turned on, wherein a temperature of the high-temperature steam generator is set to 120° C., the mixed liquid of 50% of pure water and 50% of ethanol is injected into a vaporizer of the high-temperature steam generator through the precision injection pump at 2 L/min, and the mixed liquid is carried to enter the detection system through high-purity air at 2 L/min to perform cleaning for 10 min; and then the high-temperature steam generator is turned off, and then the detection system is purged with high-purity air at 2 L/min.

(9) Toxicity effect data of this exposure is analyzed, and a fingerprint heat map of genotoxicity of exposing microorganism carrying plasmids with 29 key gene pathways to gaseous dimethyl sulfate is maded (as shown in FIG. 4). Nine of these gene pathways are significantly upregulated, indicating that after testing by the in-situ multi-toxicity endpoint detection system for VOCs, gaseous dimethyl sulfate may cause inhibition of cell division, oxidative stress, protein stress, and severe DNA damage, mutation, and recombination. In addition, as the concentration of exposure increases, cell stress and DNA structure changes become increasingly apparent, further indicating that exposure to the VOCs has great cancer risks.

Embodiment 2: Acute toxicity of two artificial gaseous pollutants (gaseous formaldehyde and gaseous phenol) is detected by using the in-situ multi-toxicity endpoint detection system for VOCs. Specific steps are the same as those in Embodiment 1 unless otherwise described.

(1) According to a detection requirement in this embodiment, the quantity of the multi-well exposure tray that need to be loaded is 1, and only one type of the hydrogel microspheres fixed with natural fluorescent microorganisms Vibrio fischeri is provided, and the quantity of hydrogel microspheres fixed with microorganisms that need to be used is 60. An initial fluorescence intensity of each hydrogel microsphere is detected and recorded, and the hydrogel microspheres are placed in the multi-well exposure tray, the multi-well exposure tray is loaded into the exposure device, and the detection system is sealed.

(2) A commercial VOCs generator is used to sequentially and accurately generate gaseous formaldehyde and phenol with nine gradient concentrations in a sealed space of 1 m³, where concentrations of formaldehyde are 5 mg/m³, 2.5 mg/m³, 1.25 mg/m³, 0.6 mg/m³, 0.3 mg/m³, 0.15 mg/m³, 0.8 mg/m³, 0.2 mg/m³, and 0.1 mg/m³; and concentrations of phenol are 50 mg/m³, 25 mg/m³, 12.5 mg/m³, 6 mg/m³, 3 mg/m³, 1.5 mg/m³, 0.8 mg/m³, 0.4 mg/m³, and 0.2 mg/m³.

(3) It is determined that concentration ratios of gaseous formaldehyde and phenol are both 10 times, the atmospheric sampling vacuum pump is turned on to collect gaseous formaldehyde and phenol in the sealed space at a particular flow rate, and adsorption of VOCs is performed in the atmospheric pre-concentrator. After adsorption of VOCs is completed, a temperature of the adsorber is increased to 300° C., a thermal desorption rate is 10 mg/min, and a thermal desorption time ranges from 15 min to 60 min.

(4) The system-controlled vacuum pump at the distal end of the detection system and the vacuum pump of the gas circulation device are turned on, and the concentrated VOCs is introduced into the exposure device at 0.5 L/min, wherein exposure is set to last 15 min, 30 min, 45 min, and 60 min; and stability of the flowmeter of the gas circulation device and the system-controlled flowmeter at the distal end of the detection system during the process is monitored.

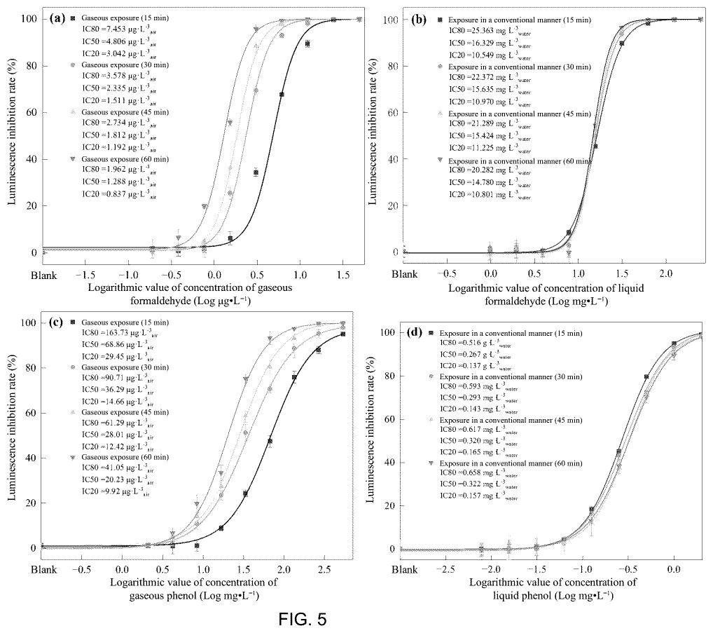

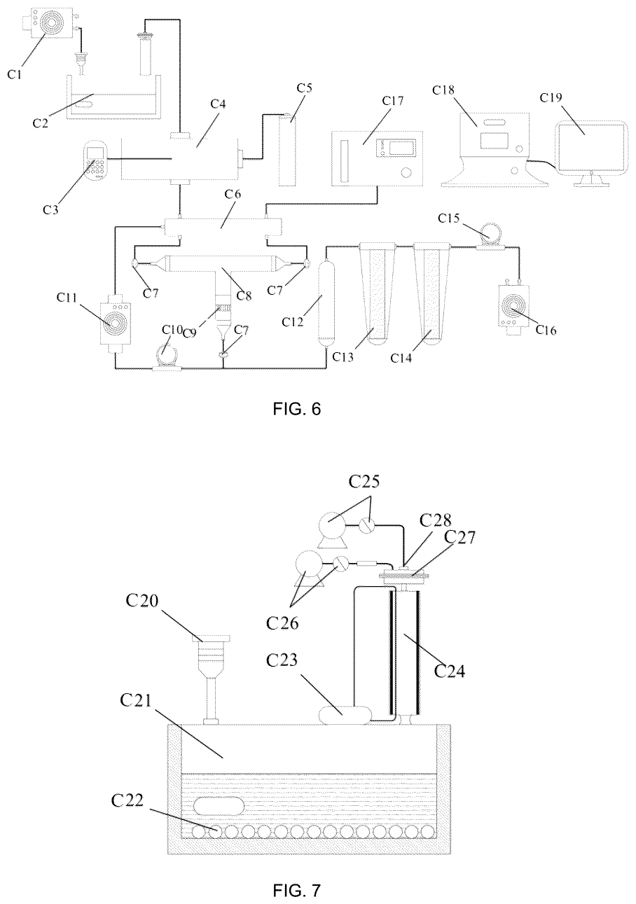

(5) After exposure to VOCs is completed, a microplate reader is used to measure final fluorescence intensity of each hydrogel microsphere, and changes of the initial fluorescence intensity and the final fluorescence intensity are obtained. A Logistic model is used to fit the obtained data, and a concentration-acute toxicity curve of the gaseous pollutant is maded, and IC50 values of acute toxicity of formaldehyde and phenol is obtained (as shown in FIG. 5).

(6) As can be learned from (a) and (b) of FIG. 5, toxicity data of gaseous formaldehyde obtained by using the in-situ multi-toxicity endpoint detection system for VOCs can be better fitted in the Logistic model, a acute toxicity curve is obtained, and a minimum detection limit of gaseous formaldehyde at concentration ratio of 10 times is 0.13 mg/m³; and compared with a conventional method for detecting acute toxicity of organic compounds, the response (an IC50 value) of exposing Vibrio fischeri to gaseous formaldehyde in a gaseous exposure process is increased by 3395 times to 11457 times.

(7) As can be learned from (c) and (d) of FIG. 5, toxicity data of gaseous phenol obtained by using the in-situ multi-toxicity endpoint detection system for VOCs can be better fitted in the Logistic model, a acute toxicity curve is formed, and a minimum detection limit of gaseous phenol at in concentration ratio of 10 times is 2.03 mg/m³; and compared with a conventional method for detecting acute toxicity of organic compounds, the response (an IC50 value) of exposing Vibrio fischeri to gaseous phenol in a gaseous exposure process is increased by 3921 times to 15818 times.

### Part Three: In-Situ Multi-Toxicity Endpoint Detection System for Atmospheric Particulate Matter

Embodiment 3: Genotoxicity of flue gas of an actual combustion source is detected by using the in-situ multi-toxicity endpoint detection system for atmospheric particulate matter constructed in the present application. Specific steps are as follows:

(1) It is ensured that all devices are in a good working state, and all necessary equipment and pipes are connected. Deionized water is added to the heating water tank of the atmospheric particulate matter concentration and accumulation device to two-thirds of the heating water tank; and ethanol is added to the condensation circulation device of the atmospheric particulate matter concentration and accumulation device, and a mixed liquid of 50% of ethanol and 50% of pure water is added to the high-temperature steam generator.

(2) According to a detection requirement in this embodiment, the quantity of the multi-well exposure tray that need to be loaded is 6, 29 types of the hydrogel microspheres fixed with recombined Escherichia coli carrying plasmids with different gene pathway plasmids are provided, and the quantity of each type of hydrogel microspheres fixed with microorganisms that need to be used is 15. An initial fluorescence intensity of each hydrogel microsphere is detected and recorded, and the hydrogel microspheres are placed in the multi-well exposure tray, the multi-well exposure tray is loaded into the exposure device, and the detection system is sealed.

(3) High-purity air is introduced to allow the high-purity air to pass through the detection system at 2 L/min, and it is determined whether values displayed by flowmeters of the detection system are the same and stable, thereby ensuring the airtightness of the detection system.

(4) An atmospheric sampling pump at a front end of the detection system is placed at 10 cm, 20 cm, and 40 cm close to a flue gas outlet of a tubular furnace with continuous combustion of pine. Atmospheric particulate matter from the combustion of pine are collected, and an concentration ratio is set to 10 times. An atmospheric particulate matter concentration laser detector is simultaneously turned on to measure a concentration of atmospheric particulate matter in a concentration control device. At different collection distances in ascending order, exposure concentrations of flue gas in the exposure device sequentially are 483.9 mg/m³, 322.6 mg/m³, and 161.3 mg/m³, and actual concentrations of flue gas from the combustion of pine are 48.39 mg/m³, 32.6 mg/m³, and 16.13 mg/m³.

(5) The system-controlled vacuum pump at the distal end of the detection system and the tubular axial-flow fan of the circulation device are turned on, and the mixed gas is introduced into the exposure device at 0.5 L/min, wherein exposure lasts 15 min; and the concentration of the atmospheric particulate matter in the atmospheric particulate matter concentration control device and stability of the electronic soap film flowmeter of the circulation device and the system-controlled flowmeter at the distal end of the detection system are continuously monitored during the process.

(6) After exposure to the atmospheric particulate matter is completed, the atmospheric particulate matter concentration and accumulation device and the atmospheric particulate matter concentration control device are turned off, pure air is introduced into the detection system for 10 min, the exposure device is turned on, and the multi-well exposure tray from the exposure device is taken out; the hydrogel microspheres fixed with microorganisms from the multi-well exposure tray are sequentially transferred into a 96 well plate, and the hydrogel microspheres are placed into the microplate reader to detect final fluorescence intensity of the hydrogel microspheres fixed with microorganisms; and changes of the initial fluorescence intensity and the final fluorescence intensity are compared and calculated to obtain toxicity effect data of this exposure.

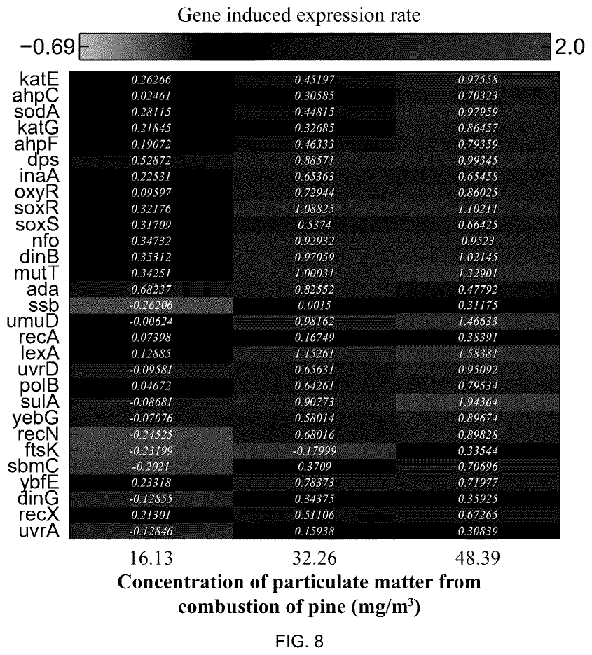

(7) Data is analyzed and a heat map of genotoxicity of atmospheric particulate matter from combustion of pine is maded. Through the in-situ multi-toxicity endpoint detection system for atmospheric particulate matter, genotoxicity of atmospheric particulate matter generated from combustion of actual pine is completely recorded. The data shows that inhibition of cell division and DNA damage may be caused by exposure to low-concentration atmospheric particulate matter from combustion of pine. As flue gas emission increases, atmospheric particulate matter from combustion of pine may induce intense DNA damage and oxidative stress in cells, and further cause DNA mutation, mispairing, and recombination, leading to carcinogenic risks. Compared with conventional toxicity collection and detection methods for pollutants, the in-situ multi-toxicity endpoint detection system for atmospheric particulate matter can assess toxicity effects of polluted air more genuinely. In summary, the fingerprint of genotoxicity of atmospheric particulate matter from combustion of pine is analyzed quickly and completely from the perspective of in-situ exposure. For details, refer to FIG. 8.

(8) After exposure to the atmospheric particulate matter is completed, ultrasonic cleaning is performed on the multi-well exposure tray for 15 min by using the mixed liquid of 50% of pure water and 50% of ethanol, then the multi-well exposure tray is loaded back to the exposure device, the exposure device is sealed, and airtightness of the detection system is checked; the high-temperature steam generator is turned on, wherein a temperature of the high-temperature steam generator is set to 120° C., the mixed liquid of 50% of pure water and 50% of ethanol is injected into the vaporizer through the precision injection pump at 2 L/min, and the mixed liquid is carried to enter the detection system through the high-purity air at 2 L/min to perform cleaning for 20 min; and then the high-temperature steam generator is turned off, and then the detection system is purged with the high-purity air at 2 L/min for 5 min.

What is claimed is:

1. An exposure device for exposing microorganisms to gaseous pollutants, comprising: a first depressurizing air inlet pipe, a second depressurizing air inlet pipe, a T-shaped air flow-mixing pipe, a multi-well exposure tray, and a pressurizing air outlet pipe,
wherein the first depressurizing air inlet pipe, and the second depressurizing air inlet pipe are configured to introduce gaseous pollutants; a structure of the first and second depressurizing air inlet pipes is funnel-shaped, a small end of the first and second depressurizing air inlet pipes is an air inlet, and a big end of the first and second depressurizing air inlet pipes is provided with threads; and diameters of the big ends of the first and second depressurizing air inlet pipes match with diameters of a left end and a right end of a horizontal pipe in the T-shaped air flow-mixing pipe, and the big ends of the first and second depressurizing air inlet pipes are respectively connected to the left end and the right end of the horizontal pipe in the T-shaped air flow-mixing pipe through the threads;
wherein the multi-well exposure tray is arranged in a vertical pipe of the T-shaped air flow-mixing pipe, and is a cylinder in which a plurality of channels are uniformly distributed; four trapezoidal support pins are arranged in each channel, and are configured to support hydrogel microspheres fixed with microorganisms, to make the hydrogel microspheres suspended in each channel; the plurality of channels are configured to provide a place for contact of the gaseous pollutants and the hydrogel microspheres; specifically, the gaseous pollutants continuously flow in the plurality of channels in the multi-well exposure tray through the first and second depressurizing air inlet pipes and are uniformly purged over surfaces of the hydrogel microspheres, to make the gaseous pollutants adhere to, accumulate at, and diffuse into the surfaces of the hydrogel microspheres, so that the microorganisms fixed in the hydrogel microspheres contact the gaseous pollutants; the hydrogel microspheres are provided with different types of microorganisms and are placed in the plurality of channels to form an array, so as to simultaneously detect multiple toxicity indicators; and the vertical pipe of the T-shaped air flow-mixing pipe and the multi-well exposure tray in the vertical pipe of the T-shaped air flow-mixing pipe form an exposure chamber;
wherein the first depressurizing air inlet pipe and the second depressurizing air inlet pipe have a structure of funnel to ensure that a velocity and a pressure of an airflow are reduced while a flow rate of the airflow is kept unchanged and to assist the T-shaped air flow-mixing pipe in uniformly distributing the airflow, so that the airflow enter from the left end and the right end of the horizontal pipe in the T-shaped air flow-mixing pipe impact each other to form a turbulence and change an airflow direction to ensure a same flow velocity and a same pressure at any position and in any cross-section of the vertical pipe of the T-shaped air flow-mixing pipe, as well as a consistent flow velocity and a consistent pressure when the airflow passing through each well in the multi-well exposure tray;
wherein the pressurizing air outlet pipe is configured to discharge the gaseous pollutants, and is also funnel-shaped; a diameter of a big opening end of the pressurizing air outlet pipe matches with a diameter of the vertical pipe of the T-shaped air flow-mixing pipe, and the big opening end and the vertical pipe are connected through threads; the pressurizing air outlet pipe and the depressurizing air inlet pipe have opposite functions; the pressurizing air outlet pipe is located at a tail end of the vertical pipe of the T-shaped air flow-mixing pipe, and generates a particular pressure at a small opening end of the pressurizing air outlet pipe, so as to ensure uniformity and stability of the airflow after passing through the multi-well exposure tray.

2. The exposure device according to claim 1, wherein the first depressurizing air inlet pipe is configured to connect to the left end of the horizontal pipe in the T-shaped air flow-mixing pipe through right-hand threads, and the second depressurizing air inlet pipe is configured to connect to the right end of the horizontal pipe in the T-shaped air flow-mixing pipe through left-hand threads.

3. The exposure device according to claim 1, wherein a range of 10 to 30 of channels is provided in the multi-well exposure tray, and a diameter of each channel ranges from 6 mm to 10 mm; and diameters of the hydrogel microspheres are smaller than diameters of the plurality of channels and range from 4 mm to 8 mm.

4. The exposure device according to claim 1, wherein multiple multi-well exposure trays are provided and are sequentially connected through threads; the multiple multi-well exposure trays are connected to each other and form a plurality of exposure layers to increase a quantity of the hydrogel microspheres that are placed in the multi-well exposure trays, thereby implementing high-flux exposure of the microorganisms to the gaseous pollutants.

5. A method for using the exposure device of claim 1, wherein the method comprises steps as follows:
(1) preparing the hydrogel microspheres fixed with microorganisms;
(2) sequentially loading the hydrogel microspheres fixed with microorganisms in the multi-well exposure tray according to a detection requirement, and detecting an initial intensity of fluorescent signals of the hydrogel microspheres fixed with microorganisms;
(3) fixing the multi-well exposure tray to a lower end of the vertical pipe of the T-shaped air flow-mixing pipe through threads, and connecting the pressurizing air outlet pipe to the T-shaped air flow-mixing pipe, wherein silicone seal gaskets are mounted at all threaded connections to ensure leakproofness of the exposure device;
(4) placing the exposure device in a detection system to make the gaseous pollutants enter the exposure device through the first and second depressurizing air inlet pipes at a flow rate ranging from 0.3 L/min to 2.0 L/min; and
(5) continuously introducing the gaseous pollutants for 15 min to 60 min, and then introducing clean air for 8 min to 10 min to completely replace the gaseous pollutants in the detection system; and sequentially placing the hydrogel microspheres in the multi-well exposure tray into a 96 well plate, and detecting a final intensity of fluorescent signals of the hydrogel microspheres fixed with microorganisms in the plurality of channels by using a microplate reader to obtain an intensity change of fluorescent signals.

6. An in-situ multi-toxicity endpoint detection system for VOCs, comprising the exposure device for exposing microorganisms to gaseous pollutants of claim 1.

7. The in-situ multi-toxicity endpoint detection system for VOCs according to claim 6, further comprising a concentration device, a distribution device, a gas circulation device, a tail gas treatment device, a detection device, and a cleaning device,
wherein the concentration device is configured to collect VOCs in air, and concentrate and accumulate VOCs until a response threshold of the in-situ multi-toxicity endpoint detection system for VOCs is reached; specifically, the concentration device comprises an atmospheric pre-concentrator and an atmospheric sampling vacuum pump that are connected, VOCs are collected and concentrated on site and introduced into the exposure device to perform toxicity effect detection; or to-be-tested air samples from a target site are collected by using a SUMMA canister, and then the SUMMA canister is loaded on the atmospheric pre-concentrator to concentrate VOCs to perform toxicity effect detection; and a concentration ratio of VOCs is made stable and controllable in a manner of adsorption-thermal desorption in cooperation with a carrier gas with a precise and controllable flow rate;
wherein the distribution device is a borosilicate glass gas manifold, in which concentrated VOCs from the concentration device and VOCs from the gas circulation device are evenly mixed to form a mixture, and the mixture enters the exposure device with equal amounts through gas circuits at two ends of the exposure device;
wherein hydrogel microspheres fixed with microorganisms for different toxicity detection are arranged in the exposure device for exposing the microorganisms to the gaseous pollutants, and the microorganisms are in full contact with the concentrated VOCs with a highly stable concentration and a highly stable flow velocity from the distribution device;
wherein the gas circulation device comprises a vacuum pump and a flowmeter that are connected, and is configured to circulate VOCs flowing out of the exposure device back to the distribution device to fully mix with the concentrated VOCs that newly enters the distribution device, thereby increasing concentration of VOCs and exposure efficiency to VOCs in the exposure device;
wherein the tail gas treatment device is a multistage treatment system, wherein a primary treatment device is a gas-washing bottle that contains 50% of pure water and 50% of ethanol and is configured to adsorb most VOCs that flows out of the exposure device; a secondary treatment device is a silica-gel drying tube, configured to remove moisture that a tail gas contains; a tertiary treatment device is two activated carbon adsorption tubes connected end to end and is configured to completely capture remaining VOCs in the tail gas; and a system-controlled flowmeter and a system-controlled vacuum pump are arranged at a distal end of the detection system, and are configured to control and stabilize an airflow rate in the detection system;
wherein the detection device is a microplate reader and is configured to detect changes in fluorescence intensity of the hydrogel microspheres fixed with microorganisms before and after exposure; and
wherein the cleaning device is located at an upstream of the distribution device, is a high-temperature steam generator equipped with a high-purity air carrier gas, and generates high-temperature steam containing 50% of ethanol and 50% of pure water; the high-temperature steam is carried by the high-purity air carrier gas to purge the detection system after toxicity detection is completed.

8. The in-situ multi-toxicity endpoint detection system for VOCs according to claim 7, wherein the exposure device, the concentration device, the distribution device, the gas circulation device, the tail gas treatment device, the detection device, and the cleaning device are connected through metal pipes with a diameter of 10 mm, the detection system is an absolutely sealed system, all connections of the detection system are sealed by silicone seal gaskets to ensure airtightness of the detection system, so as to avoid secondary pollution to environment and adverse health effects on testing personnel caused by leakage of concentrated VOCs.

9. The in-situ multi-toxicity endpoint detection system for VOCs according to claim 8, wherein three air inlets of the distribution device are respectively connected to the atmospheric pre-concentrator, a distal end of the gas circulation device, and the high-temperature steam generator; two air outlets of the distribution device are respectively connected to the first and second depressurizing air inlet pipes of the exposure device; the pressurizing outlet pipe of the exposure device is connected to a first end of a three-way pipe, a second end of the three-way pipe is connected to the flowmeter of the gas circulation device which is then connected to the vacuum pump of the gas circulation device to circulate VOCs to the distribution device, and a third end of the three-way pipe is connected to the gas-washing bottle of the tail gas treatment device; the gas-washing bottle, the silica-gel drying tube, the two activated carbon adsorption tubes, the system-controlled flowmeter, and the system-controlled vacuum pump are sequentially connected.

10. The in-situ multi-toxicity endpoint detection system for VOCs according to claim 9, wherein a working procedure of the detection system is as follows:
(1) selecting an appropriate adsorption material according to physical and chemical properties of VOCs contained in to-be-tested air samples, filling the adsorption material in an adsorber of the atmospheric pre-concentrator, and adding a mixed liquid of 50% of ethanol and 50% of pure water to the gas-washing bottle and the high-temperature steam generator;
(2) determining, according to a detection requirement, a quantity of the multi-well exposure trays that need to be loaded and a type and a quantity of the hydrogel microspheres that need to be used; detecting and recording an initial fluorescence intensity of each hydrogel microsphere; placing the hydrogel microspheres in the multi-well exposure tray, loading the multi-well exposure tray into the exposure device, and sealing the detection system;
(3) introducing air to allow the air to pass through the detection system at 2 L/min, and determining whether values displayed by flowmeters of the detection system are the same and stable, thereby ensuring the airtightness of the detection system;
(4) determining the concentration ratio of VOCs, and turning on the atmospheric sampling vacuum pump to collect VOCs in air of a target site at a particular flow rate, or connecting the SUMMA canister to the atmospheric pre-concentrator to concentrate VOCs in a pre-collected air sample; after adsorption of VOCs is completed, increasing a temperature of the adsorber to 200° C. to 600° C. to make VOCs adsorbed and concentrated in the adsorption material into a gaseous state, wherein a thermal desorption time ranges from 15 min to 60 min, mixing air and VOCs in the gaseous state; and obtaining a mixed gas containing VOCs with an accurate and controllable concentration ratio by controlling a rate of thermal desorption and a flow rate of the carrier gas;
(5) turning on the system-controlled vacuum pump at the distal end of the detection system and the vacuum pump of the gas circulation device to introduce the concentrated VOCs into the exposure device at 0.5 L/min to 2.0 L/min, wherein exposure lasts 15 min to 60 min; and monitoring stability of the flowmeter of the gas circulation device and the system-controlled flowmeter at the distal end of the detection system during the process;
(6) after exposure to VOCs is completed, introducing pure air into the detection system for 8 min to 12 min, taking out the multi-well exposure tray from the exposure device and transferring the hydrogel microspheres from the multi-well exposure tray into a 96 well plate, and detecting final fluorescence intensity of each hydrogel microsphere by using the microplate reader; comparing and calculating changes of the initial fluorescence intensity and the final fluorescence intensity to obtain toxicity effect data of this exposure; and
(7) after exposure to VOCs is completed, performing ultrasonic cleaning on the multi-well exposure tray for 15 min to 30 min by using the mixed liquid of 50% of pure water and 50% of ethanol, then loading the multi-well exposure tray back to the exposure device, and sealing and checking the airtightness of the detection system; turning on the high-temperature steam generator, wherein a temperature of the high-temperature steam generator is set to 110° C. to 130° C., injecting the mixed liquid of 50% of pure water and 50% of ethanol into a vaporizer of the high-temperature steam generator through a precision injection pump at 2 L/min to 4 L/min, and carrying the mixed liquid to enter the detection system through air at 2 L/min to 4 L/min to perform cleaning for 10 min to 20 min; and then turning off the high-temperature steam generator, and purging the detection system with high-purity air at 2 L/min to 4 L/min for 5 min to 10 min.

11. An in-situ multi-toxicity endpoint detection system for atmospheric particulate matter, comprising the exposure device for exposing microorganisms to gaseous pollutants of claim 1.

12. The in-situ multi-toxicity endpoint detection system for atmospheric particulate matter according to claim 11, further comprising an atmospheric particulate matter concentration and accumulation device, an atmospheric particulate matter concentration control device, a gas distribution device, a circulation device, and a tail gas treatment device,
wherein the atmospheric particulate matter concentration and accumulation device comprises an atmospheric sampling pump, an atmospheric particulate matter sampler, a heating water tank, a condensation circulation device, and a virtual impaction head;
wherein the atmospheric particulate matter sampler is provided with three impaction heads of PM2.5, PM5, and PM10, to collect atmospheric particulate matter with different particle sizes in air; under an action of a suction force of the atmospheric sampling pump, air samples are collected, and after being sorted by the three impaction heads, the atmospheric particulate matter of particular particle sizes enters the heating water tank; and a heating rod is arranged in the heating water tank to heat deionized water to 40 degrees Celsius to 50 degrees Celsius to generate sufficient water vapor to obtain atmospheric particulate matter in a saturated state;
the atmospheric particulate matter in a saturated state enters the condensation circulation device at a flow rate of 30 L/min to 50 L/min in a main gas circuit, and a temperature in the condensation circulation device ranges from −18 degrees Celsius to 25 degrees Celsius, so that the atmospheric particulate matter in a saturated state gradually condense and grow large in a condensation tube to eventually form large atmospheric particulate matter with particle diameters ranging from 3 micrometers to 4 micrometers; and eventually the large atmospheric particulate matter passes through the virtual impaction head at a flow rate of 1 L/min to 6 L/min in a concentration gas circuit, exits from the atmospheric particulate matter concentration and accumulation device at a nozzle at an increased speed, and enters the atmospheric particulate matter concentration control device;
wherein the atmospheric particulate matter concentration control device provides a closed space and comprises an atmospheric particulate matter concentration laser detector and a high-purity compressed air cylinder; the atmospheric particulate matter concentration laser detector is configured to continuously monitor a concentration of the atmospheric particulate matter that flows from the atmospheric particulate matter concentration and accumulation device; and it is determined, according to a concentration value of the atmospheric particulate matter, whether to increase an accumulation ratio of the atmospheric particulate matter concentration and accumulation device, or open the high-purity compressed air cylinder, to precisely control a flow rate of a high-purity air carrier gas that is introduced into the atmospheric particulate matter concentration control device; mixing of the high-purity air carrier gas with the atmospheric particulate matter is performed to obtain a mixed gas, thereby implementing precise control of a concentration of the atmospheric particulate matter, and determining a conversion relationship between the concentration of the atmospheric particulate matter that the multi-well exposure tray is exposed to and an actual concentration of the atmospheric particulate matter in air;
wherein the gas distribution device is configured to distribute the mixed gas containing the atmospheric particulate matter with an accurate concentration from the atmospheric particulate matter concentration control device, fully and uniformly mix the mixed gas with the atmospheric particulate matter circulated back from the circulation device to obtain a mixture, and enable the mixture of the mixed gas and the atmospheric particulate matter circulated back from the circulation device to enter the exposure device with equal amounts through the first and second depressurizing air inlet pipe; and specifically, the gas distribution device is a borosilicate glass gas distribution tube;
wherein the hydrogel microspheres fixed with microorganisms for different toxicity detection are arranged in the exposure device for exposing the microorganisms to the gaseous pollutants, and are in full contact with the mixture with a highly stable concentration and a highly stable flow rate from the gas distribution device, to expose the microorganisms to the atmospheric particulate matter;
wherein the circulation device comprises a tubular axial-flow fan and an electronic soap film flowmeter; the atmospheric particulate matter that flows out of the exposure device is circulated back to the gas distribution device and is fully mixed with the mixed gas containing the atmospheric particulate matter that enters the gas distribution device to increase a concentration of the atmospheric particulate matter and exposure efficiency to the atmospheric particulate matter in the exposure device; and
wherein the tail gas treatment device is a multistage treatment system; wherein a primary treatment device is a silica-gel drying tube, configured to adsorb moisture absorbed by the atmospheric particulate matter in a concentration and accumulation process; a secondary treatment device and a tertiary treatment device are two atmospheric particulate matter filters that are connected end to end, and are configured to avoid secondary pollution caused by discharge of the atmospheric particulate matter in the detection system to environment; and a system-controlled flowmeter and a system-controlled vacuum pump are arranged at a distal end of the detection system, and are configured to control and stabilize an airflow rate in the detection system.

13. The in-situ multi-toxicity endpoint detection system for atmospheric particulate matter according to claim 12, wherein the multi-well exposure tray in the exposure device is designed as a detachable structure, so that quick assembly and disassembly can be implemented.

14. The in-situ multi-toxicity endpoint detection system for atmospheric particulate matter according to claim 12, further comprising a detector, the detector is a microplate reader; after exposure of the multi-well exposure tray to the atmospheric particulate matter is completed, the multi-well exposure tray in the exposure device is quickly disassembled, the hydrogel microspheres fixed with microorganisms in the multi-well exposure tray are placed into a 96 well plate, and each hydrogel microsphere in the 96 well plate is subjected to the microplate reader for fluorescence intensity detection.

15. The in-situ multi-toxicity endpoint detection system for atmospheric particulate matter according to claim 14, further comprising a cleaning device, wherein the cleaning device is located upstream of the gas distribution device, and comprises a high-temperature steam generator equipped with an air carrier gas; after exposure of the multi-well exposure tray to the atmospheric particulate matter is completed, the exposure device is easily disassembled using a quick-connect buckle provided on the exposure device; the exposure device then is placed in an ultrasonic cleaning machine that contains 50% of ethanol and 50% of pure water to perform ultrasonic cleaning for 15 min to 30 min, followed by loading back into the exposure device, sealing, and airtightness checking; the high-temperature steam generator is turned on, wherein a temperature of the high-temperature steam generator is set to 110° C. to 130° C., a mixed liquid of 50% of pure water and 50% of ethanol is injected into a vaporizer through a precision injection pump at 2 L/min to 4 L/min, and the mixed liquid is carried to enter the detection system through air at 2 L/min to 4 L/min to perform cleaning for 10 min to 20 min; and then the high-temperature steam generator is turned off, and the detection system is purged with air at 2 L/min to 4 L/min for 5 min to 10 min.

16. The in-situ multi-toxicity endpoint detection system for atmospheric particulate matter according to claim 12, wherein the atmospheric particulate matter concentration and accumulation device, the atmospheric particulate matter concentration control device, the gas distribution device, the circulation device, and the tail gas treatment device in the detection system are connected through metal pipes with a diameter of 10 mm, the detection system is an absolutely sealed system, and all connection are sealed by silicone seal gaskets to ensure airtightness of the detection system, so as to avoid secondary pollution to environment and adverse health effects on testing personnel caused by leakage of concentrated atmospheric particulate matter.

17. The in-situ multi-toxicity endpoint detection system for atmospheric particulate matter according to claim 16, wherein a connection manner in the detection system is as follows: the atmospheric particulate matter concentration and accumulation device is connected to a first air inlet of the atmospheric particulate matter concentration control device, a second air inlet of the atmospheric particulate matter concentration control device is connected to the high-purity compressed air cylinder, and a probe of the atmospheric particulate matter concentration laser detector is fixed on an inner wall of the atmospheric particulate matter concentration control device; an outlet of the atmospheric particulate matter concentration control device is connected to a first air inlet of the gas distribution device; a second and a third air inlets of the gas distribution device are respectively connected to a distal end of the circulation device and the high-temperature steam generator; a first and a second air outlets of the gas distribution device are connected to the first and second depressurizing air inlet pipes of the exposure device through the quick-connect buckles; the pressurizing air outlet pipe of the exposure device is connected to a three-way pipe through the quick-connect buckle, and one remaining end of the three-way pipe is connected to the electronic soap film flowmeter of the circulation device that is connected to the tubular axial-flow fan, wherein the tubular axial-flow fan is connected to the gas distribution device; the pressurizing air outlet pipe of the exposure device is also connected to the silica-gel drying tube of the tail gas treatment device; and the silica-gel drying tube, the atmospheric particulate matter filters, the system-controlled flowmeter, and the system-controlled vacuum pump are sequentially connected.

18. The in-situ multi-toxicity endpoint detection system for atmospheric particulate matter according to claim 17, wherein a working procedure is as follows:
(1) adding deionized water to the heating water tank of the atmospheric particulate matter concentration and accumulation device to two-thirds of the heating water tank; adding ethanol to the condensation circulation device of the atmospheric particulate matter concentration and accumulation device, and adding a mixed liquid of 50% of ethanol and 50% of pure water to the high-temperature steam generator;
(2) determining, according to a detection requirement, a quantity of the multi-well exposure tray that need to be loaded and a type and a quantity of the hydrogel microspheres fixed with microorganisms that need to be used; detecting and recording an initial fluorescence intensity of each hydrogel microsphere; placing the hydrogel microspheres in the multi-well exposure tray, and loading the multi-well exposure tray into the exposure device, and sealing the detection system;
(3) introducing high-purity air to allow the high-purity air to pass through the detection system at 2 L/min, and detecting whether values displayed by flowmeters of the detection system are the same and stable, thereby ensuring the airtightness of the detection system;
(4) determining a concentration ratio of the atmospheric particulate matter, turning on the atmospheric sampling pump, and collecting the atmospheric particulate matter in air of a target site; after the atmospheric particulate matter enter the atmospheric particulate matter concentration and accumulation device, starting a concentration procedure; and continuously monitoring changes in a concentration of the atmospheric particulate matter in the atmospheric particulate matter concentration control device, and adjusting the accumulation ratio of the atmospheric particulate matter concentration and accumulation device, or introducing air with precise flow rate to mix with the atmospheric particulate matter to obtain the mixed gas, until the concentration of the atmospheric particulate matter in the atmospheric particulate matter concentration control device meets the detection requirement;
(5) turning on the system-controlled vacuum pump at the distal end of the detection system and the tubular axial-flow fan of the circulation device, and introducing the mixed gas into the exposure device at 0.5 L/min to 2.0 L/min, wherein exposure lasts 15 min to 60 min; and continuously monitoring the concentration of the atmospheric particulate matter in the atmospheric particulate matter concentration control device and stability of the electronic soap film flowmeter of the circulation device and the system-controlled flowmeter at the distal end of the detection system during the process;
(6) after exposure to the atmospheric particulate matter is completed, turning off the atmospheric particulate matter concentration and accumulation device and the atmospheric particulate matter concentration control device, introducing pure air into the detection system for 10 min to 20 min, taking out the multi-well exposure tray from the exposure device and transferring the hydrogel microspheres fixed with microorganisms from the multi-well exposure tray into a 96 well plate, and detecting final fluorescence intensity of the hydrogel microspheres fixed with microorganisms by using the microplate reader; comparing and calculating changes of the initial fluorescence intensity and the final fluorescence intensity to obtain toxicity effect data of this exposure; and
(7) after exposure to the atmospheric particulate matter is completed, performing ultrasonic cleaning on the multi-well exposure tray for 15 min to 30 min by using the mixed liquid of 50% of pure water and 50% of ethanol, then loading the multi-well exposure tray back to the exposure device, and sealing and checking airtightness of the detection system; turning on the high-temperature steam generator, wherein a temperature of the high-temperature steam generator is set to 110° C. to 130° C., injecting the mixed liquid of 50% of pure water and 50% of ethanol into the vaporizer through the precision injection pump at 2 L/min to 4 L/min, and carrying the mixed liquid to enter the detection system through the high-purity air at 2 L/min to 4 L/min to perform cleaning for 10 min to 20 min; and then turning off the high-temperature steam generator, and purging the detection system with the high-purity air at 2 L/min to 4 L/min for 5 min to 10 min.
